(19) United States
(12) Patent Application Publication
(10) Pub. No.: US 2024/0024191 A1
(43) Pub. Date: Jan. 25, 2024

(54) HardeR

(71) Applicant: Abraham Fernando Carpio, Hyattsville, MD (US)

(72) Inventor: Abraham Fernando Carpio, Hyattsville, MD (US)

(73) Assignee: Carpio Fernando Abraham, Hyattsville, MD (US)

(21) Appl. No.: 18/340,114

(22) Filed: Jun. 23, 2023

(51) Int. Cl. A61H 19/00; A61H 11/00; A61F 6/04

(52) U.S. Cl. CPC A61H 19/32; A61H 19/50; A61H 11/00; A61F 6/04; A61H 2011/005; A61H 2205/087; A61H 2201/0161; A61F 2006/047

(57) ABSTRACT

This invention concerns condoms, prophylactics and contraceptive devices made of silicone rubber having a body with one open end and a closed end. The open end is fused with a rubber band or hereinafter “ligature” that goes over the scrotum and around the back of the male testicles to act as a scrotal fastener for the condom and testicular stimulator, which during intercourse will fasten the condom to the shaft of the penis from the base of the scrotum, to (i) prevent slippage, and (ii) generate increased ejaculatory/orgasmic sensation.

### FIELD OF THE INVENTION

This invention concerns a condom commonly used for contraceptive and prophylactic use. However, this invention improves the insertive condom for use in vaginal or anal intercourse, to prevent slippage and loss, which without proper fastening can increase the risk of sexually transmitted diseases, and solves this risk by securing the condom to the shaft of the male penis, through the addition of a scrotum ligature which will also enhance the sexual experience and sensation of orgasm.

### BACKGROUND OF THE INVENTION

This invention aims to overcoming problems associated with traditional un-fastened condoms in the fight against sexually transmitted diseases, including the spread of the human immunodeficiency virus (HIV) that can result in acquired immune deficiency syndrome (AIDS), COVID 19 and other STD's, since the invention fastens the condom opening to the base of the penis with a ligature over the testicles to prevent condom loss or displacement during intercourse.

As it is well known, increased protection from sexually transmitted diseases is a public health concern that affects all people, regardless of sexual orientation, nationality and age group. The COVID 19 pandemic brought additional focus to sexually , transmissible viruses, given its lethality among certain groups of sexually fit persons, that otherwise have increased vulnerability to this virus. The proper use of condoms is thus strongly advocated to combat the transmission of HIV, STD's and COVID 19.

While condoms are produced to serve male and female use, they also come in hybrid varieties for use by both types of partners, regardless of sexual orientation.

As it is known, the traditional male condom has an elongated tubular sheath, typically made of latex or polyurethane, or some type of fine animal skin.

The application and use of this condom as well as the conventional condom is effected by unrolling, stretching and pulling the sheath onto the erect penis. In some varieties, the open end generally has a peripheral portion that may function as a constricted rim to prevent the condom from losing fluid. The condom is conventionally made in this fashion, with the expectancy of the condom to remain in place by friction of the unrolled section of the opening, or through pressure against the material during intercourse.

Need for fastener to improve fastening in place, prevent loss and increase sensation As it is now designed, the tubular sheath of the male condom is open at one end for insertion of the penis and remains closed at the other end to trap ejaculate of seminal fluids.

As it is currently used and designed, the standard male condom is elastically fitted to the penis, with the expectancy that during coitus it remains outstretched and taut against the skin of the male through friction and pressure.

But this design generates two problems. First, relying on plain friction produced by pressure can increase the hazard of the condom being torn during use. And second, the integrity of the condom can also he compromised by slippage and loss of the unit during intercourse, and most likely when tumescence recedes after climax. In these latter instances, as the tumescence recedes, the normal condom will lose friction and the condom will likely slip into the vaginal cavity or anal cavity.

It is for these reasons that standard male condoms are well known to be only partially effective in the prevention of sexually transmitted diseases, such as HIV, given the potential for leakage and loss.

Studies have shown that commercial thin membrane latex male condoms can have a failure rate up to 17%. These traditional male condoms have thus now acquired an unacceptably high risk of failure, but none greater than full slippage.

Upon slippage, the entire unit of protection being lost, both partners are exposed to unwanted pregnancies (which is more serious now that Roe v. Wade was overturned) and STD transmission.

Importantly, another great disadvantage of the traditional male condom is the loss of sensation experienced by the active male caused by tight-fitting in order to rely on friction for the condom to stay in place. Therefore, the conventional condom does not permit adequate sensitivity for the active male during sexual intercourse because it sacrifices sensation for increased friction. The scrotal ligature condom solves these failings.

### SUMMARY OF THE INVENTION

In light of the foregoing, the object of the present invention to provide a condom that is safer than currently available commercial condoms, and eliminate the sacrifice of friction for sensation, with increased security and sensation.

The ultimate goal is to provide an improved condom that can be applied before intercourse so as not to interrupt the sexual activity, Which (i) won't slip during intercourse, (ii) reduce pressure-based-friction on the shaft; (iii) and which will improve scrotal stimuli for the active male, while (iv) preventing the transmission of STDs or unwanted pregnancies upon loss of the unit.

In furtherance of the objects of the present invention, a condom according to an embodiment of this invention has a sheath body with an open end and a closed end, and a fused ligature at the open end to wrap the scrotum and testicles for security and increased sensation.

The sheath body of the condom is standard, with the exception of the base-fused ligature that will secure the shaft in place and prevent slippage during intercourse.

Further, the fused-based ligature will not constrict the erect penis shaft as condom would to achieve friction, but rather rely on the ligature to enhance scrotal sensation during sexual activity.

Viewed in its preferred embodiment, the condom is folded flat as a normal condom, and the ligature exposed only upon unrolling, to facilitate packaging.

But in order to make a condom according to the objectives of the present invention, the condom is preferably made from normal latex or silicone so that based-fused ligature is sufficiently attached to the open end, and withstand the encirclement of the testicles.

This invention will thus provide a more comfortable fit for the active partner as it loosely accommodates the penis, so that movement of the penis is possible during coitus and remains fastened to the testicles.

While the above description of the present invention in relation to the drawings and embodiments is accurate, it is to be understood that the description is not intended to limit the scope of the invention to the drawings or embodiments described herein. On the contrary, the description is intended to cover alternative modifications and equivalents that may or may not be included in the spirit and the scope of the invention as defined by the appended claims.

### DETAILED DESCRIPTION OF THE INVENTION

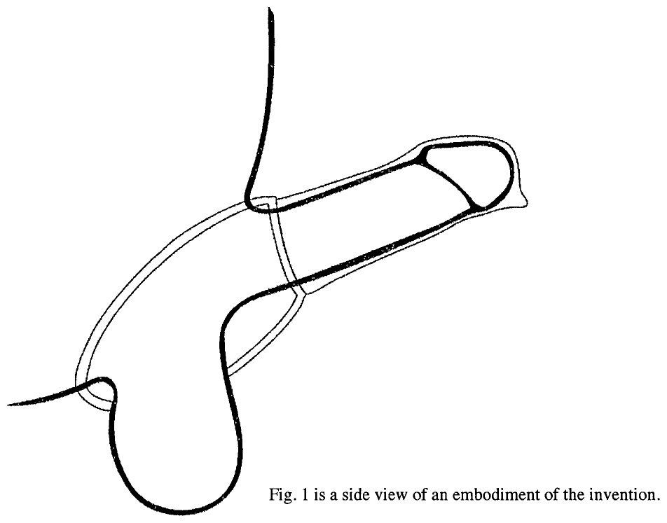

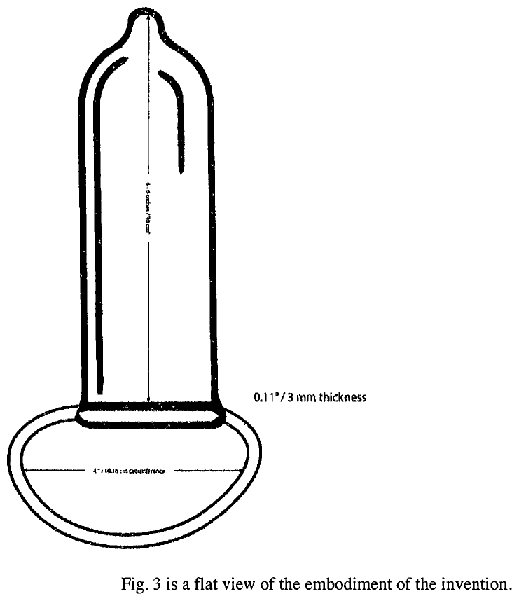

As shown in FIGS. 1, 2 and 3, the condom 1 of the present invention has a sheath body with a proximal end that is open and a distal end that is closed. In a preferred embodiment, the open end has a diameter of approximately 2.0 inch, although the opening can expand to at least three times that diameter due to the condom being made of stretchable material, such as heavy gauge silicone rubber.

At the open end, the condom has a first rubber-band or ligature base of 4 inches in diameter.

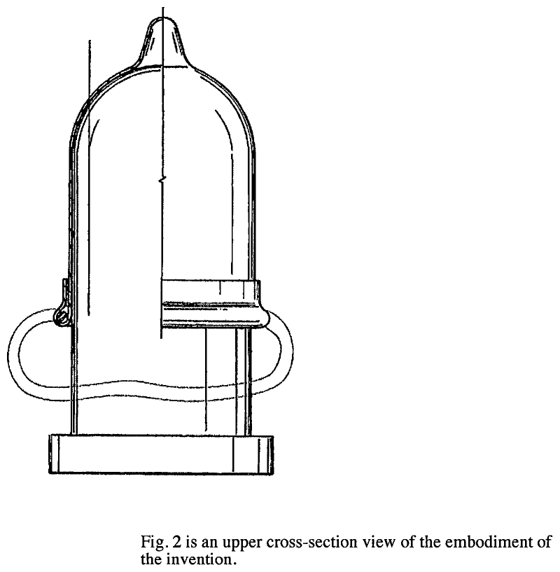

In a preferred embodiment, the first collar is defined by an annular rim that is turned towards the sheath body to encase the rubber-hand as shown in FIG. 2.

In an embodiment of the invention, the rubber-hand is encased in the folding of the condom, and unfolded to be revealed and extended over the scrotum, as shown in FIG. 1.

The rubber-band or ligature serves to fasten the condom in place, prevent slippage, strengthen the friction of the sheath and minimizes movement or twisting during sexual activity.

The rubber-band or ligature also enables and promotes scrotal stimuli by virtue of its friction and tension on the scrotal area, and in testing several subjects report enhanced ejaculation sensation and pleasure at climax.

In a preferred embodiment of the invention, the heavy gauge silicone is at least 0.06 inches thick, so that the rubber-band has sufficient structural rigidity to enable vaginal and anal insertion without loss or slippage of the unit.

To use condom 1 for vaginal or anal intercourse, for example, condom is placed on the erect penis, and the rubber band or ligature is placed over the scrotal area (the testicles), and the closed end is inserted into the vagina or anal cavity.

1. A condom comprising of a body with an open end and a closed end, and a rubber band ligature fused and attached to the open end.

1A. Said body will extend along the erect longitudinal axis of the penis. The open end will encase and fold the rubber band as it folds for packaging, and unfold to reveal the band.

1B. The advantage of the band is for the user to elastically extend it over the scrotum and circumference of the testicles to accomplish two goals. First, to fasten the condom and prevent slippage or loss during vaginal or anal intercourse; and second to stimulate the scrotal nerves by slight tension and pressure over the scrotal area in contact with the band.

1C. Wherein said condom is made of silicone rubber such as to enable the unfolding over the erect penis; and well as the band, to extend over the scrotum.
